(19) United States
(12) Patent Application Publication
(10) Pub. No.: US 2018/0095091 A1
(43) Pub. Date: Apr. 5, 2018

(54) METHOD OF DIGITIZING NUTRITIONAL STATUS, MUSCLE TURNOVER, AND RISK ASSESSMENT

(71) Applicant: Chang Gung Memorial Hospital, Keelung, Keelung (TW)

(72) Inventor: CHAO-HUNG WANG, Keelung (TW)

(21) Appl. No.: 15/488,985

(22) Filed: Apr. 17, 2017

(30) Foreign Application Priority Data
Oct. 5, 2016 (TW) 105132258

(51) Int. Cl. G01N 33/68; G01N 33/574; G06F 19/00; A61B 5/00

(52) U.S. Cl. CPC G01N 33/6812; G01N 2800/02; G06F 19/3431; G06F 19/3475; A61B 5/4866; A61B 5/4872; A61B 5/4519; A61B 5/7275; G01N 2800/50; G01N 2800/7066; G01N 2800/122; G01N 2800/26; G01N 2800/32; G01N 2800/347; G01N 2800/7042; G01N 2800/042; G01N 2800/321; G01N 33/574

(57) ABSTRACT

The present invention provides a method of digitizing nutritional status, muscle turnover, and risk assessment using the formulas containing four amino acids, which are Histidine, Leucine, Ornithine and Phenylalanine. The method of present invention can provide information regarding personalized nutritional status, muscle turnover and risk assessment, understand the effect of nutrition intervention, assist muscle growth and the effect of rehabilitation training, and improve patients' quality of life and bodily function.

### CROSS-REFERENCE TO RELATED APPLICATION

This application claims the priority of Taiwanese patent application No. 105132258, filed on Oct. 5, 2016, which is incorporated herewith by reference.

### BACKGROUND OF THE INVENTION

1. Field of the Invention

The present invention is to provide a method of assessing nutritional status, muscle turnover, and risk assessment, and more particularly, to provide a method of digitizing nutritional status, muscle turnover, and risk assessment.

2. The Prior Arts

With regard to nutrition care, balanced nutrition come from the food is the best way the world recognized. However, personalized nutrition intervention has aroused worldwide concern due to the individual differences, especially in the state of diseases.

For critical ill patients, they usually face the problem of over (overfeeding) or under (underfeeding) nutritional supplement. Most people think that critical ill patients need to supply more energy, but without a scientific assessment tool, they may be overfed; however, they may also be seriously underfed. It is also difficult to ensure that the nutrition we are supplying is what the patients need. For example, when caring for critical ill patients, we also want to know the information about muscle lysis (protein breakdown), whether the calories energy is normally produced or abnormally produced by taking muscle as the energy source, whether body's metabolites cause liver overloaded, the level of amino acids in the body over or inadequate, and whether we need to encourage patients to take appropriate rehabilitation to change metabolism. The information regarding assessments described above needs to be obtained at the same time and to be integrated for interpretation; however, such an assessment platform does not exist at the present time.

Furthermore, the clinical requirement of high-quality nutrition assessment and intervention is not only for critical ill patients, but also for the patients recovering after surgery, having a healing wound, aging, suffering from cancer under chemotherapy, chronic lung disease, chronic kidney disease, cardiovascular disease, undergoing kidney dialysis, lung disease, etc. The quality of nutrition assessment and intervention has a close relationship with the improvement in disease status and prognosis. For example, in patients with chronic lung disease, recurrent dyspnea may cause the use of steroid and finally lead to cachexia, which result in muscle lysis, liver congestion, massive loss of muscle mass leading into a vicious cycle. They need aggressive rehabilitation exercise training and appropriate nutrient supply to prevent muscle lysis. However, so far, we do not have a digitally integrated and advanced nutrition assessment platform to evaluate how many calories and protein need to be supplied, whether muscle lysis is adequately reduced in response to supplied nutrients and rehabilitation exercise training, whether nutrients are over-supplied leading to liver overloaded, and whether the patient status is progressing or regressing.

Patients with chronic kidney disease are usually suggested to take a low-protein diet, but it is difficult to know whether their body is in a status of severe amino acid deficiency based on current nutrition assessment tools; thus, patients with chronic kidney disease generally have a poor prognosis. For patients recovering after surgery, they are suggested to take dietary supplements or expensive amino acids, but their nutritional status is actually not adequately estimated and it is unknown whether they are overfed or underfed. Thus, the critical point is how to provide nutrition intervention appropriately on demand and grasp the golden period of wound repairing. For nutritional aspects of the elderly, due to aging, they do not regenerate muscle easily, which leads to an increased risk of sarcopenia, fall-related fractures due to sarcopenia, and an increased burden of long-term care. However, actually, in some elderly, the amount of amino acids in their body is far from adequate to generate muscle. It is critical to supply them with appropriate nutrients based on exquisite assessments, and to provide them with appropriate rehabilitation training according to the parameters of muscle lysis assessed regularly. All these reasons indicate the importance of a digitally integrated and advanced nutrition assessment platform.

### SUMMARY OF THE INVENTION

As such, a primary objective of the present invention is to provide a method of digitizing nutritional status, muscle turnover, and risk assessment in a subject, comprising the steps of:
(a) measuring the level of an amino acid selected from the group consisting of Histidine, Leucine, Ornithine and Phenylalanine in a biological sample of the subject using a detecting method;
(b) calculating nutritional status and risk scores of the subject using formulas as follows:
   (1) Formula 1 (muscle metabolism turnover): Histidine level/Phenylalanine level;
   (2) Formula 2 (digitized nutritional status score): (−19.265 to −15.763)×(the value of Formula 1)+(0.059 to 0.073)×Ornithine level+(18.776 to 22.948);
   (3) Formula 3 (liver metabolic function): the value of Formula 3 calculated based on gender is corrected Ornithine level (Oc):
      Male:
      if Leucine level≤[(mean of Leucine level in normal male blood)−(standard deviation of Leucine level in normal male blood)],
      Oc=[Ornithine level×[(mean of Leucine level in normal male blood)−(standard deviation of Leucine level in normal male blood)]]/Leucine level;
      if Leucine level>[(mean of Leucine level in normal male blood)-(standard deviation of Leucine level in normal male blood)],
      Oc=Ornithine level.
      Female:
      if Leucine≤[(mean of Leucine level in normal female blood)−(standard deviation of Leucine level in normal female blood)],
      Oc=[Ornithine level×[(mean of Leucine level in normal female blood)−(standard deviation of Leucine level in normal female blood)]]/Leucine level;
      if Leucine level≤[(mean of Leucine level in normal female blood)−(standard deviation of Leucine level in normal female blood)],
      Oc=Ornithine level;
   (4) Formula 4 (muscle lysis): the value of Formula 4 calculated based on gender is corrected Phenylalanine level (Pc):
      Male:
      if Leucine level≤[(mean of Leucine level in normal male blood)−(standard deviation of Leucine level in normal male blood)],
      Pc=[Phenylalanine level×[(mean of Leucine level in normal male blood)−(standard deviation of Leucine level in normal male blood)]]/Leucine level;
      if Leucine level>[(mean of Leucine level in normal male blood)−(standard deviation of Leucine level in normal male blood)],
      Pc=Phenylalanine level;
      Female:
      if Leucine level≤[(mean of Leucine level in normal female blood)−(standard deviation of Leucine level in normal female blood)],
      Pc=[Phenylalanine level×[(mean of Leucine level in normal female blood)−(standard deviation of Leucine level in normal female blood)] ]/Leucine level;
      if Leucine level>[(mean of Leucine level in normal female blood)−(standard deviation of Leucine level in normal female blood)],
      Pc=Phenylalanine level; and
   (5) Formula 5 (metabolite loading to liver)=−[(−1.414 to −1.157)×Ornithine level/(Leucine level+Histidine level-Phenylalanine level)+(0.0063 to 0.0077)×(the value of Formula 3)]×(9 to 11), all formulas can be modified according to requirement,
(c) interpreting the nutritional status of the subject,
   wherein the value obtained from Formulas 1 and 2 is the nutritional status score, which is compared with the reference value at x-axis of the map of digitizing nutritional status, muscle turnover, and risk assessment (FIG. 2), the scale of the nutritional status score is divided as N, A, B, C and D, N representing normal, A representing early abnormal, B representing significantly abnormal but no symptom, C representing very abnormal, D representing extremely abnormal; the numerical range of N is calculated by a normal subject, and then the numerical range of A, B, C and D are separately defined based on female and male,
   Female:
   the lower limit of 95% confidence interval of N≤N≤the upper limit of 95% confidence interval of N;
   the lower limit of 95% confidence interval of N+4.42≤A≤the lower limit of 95% confidence interval of N+5.96;
   the lower limit of 95% confidence interval of N+7.06≤B≤the lower limit of 95% confidence interval of N+9.01;
   the lower limit of 95% confidence interval of N+11.75≤C≤the lower limit of 95% confidence interval of N+13.52;
   the lower limit of 95% confidence interval of N+18.07≤D≤the lower limit of 95% confidence interval of N+23.66;
   Male:
   the lower limit of 95% confidence interval of N≤N≤the upper limit of 95% confidence interval of N;
   the lower limit of 95% confidence interval of N+3.3≤A≤the lower limit of 95% confidence interval of N+4.46;
   the lower limit of 95% confidence interval of N+6.96≤B≤the lower limit of 95% confidence interval of N+7.64;
   the lower limit of 95% confidence interval of N+11.36≤C≤the lower limit of 95% confidence interval of N+12.35;
   the lower limit of 95% confidence interval of N+18.04≤D≤the lower limit of 95% confidence interval of N+20.77;
   the value obtained from the combination of the concentration of Leucine and Formulas 3 to 5 is the risk score, which is compared with the reference value at y-axis of the map of digitizing nutritional status, muscle turnover, and risk assessment to assess risk level from A0 to A8 or from A0 to A-5, which represent gradually higher risks; A0 represents lowest-risk, A8 and A-5 represents highest-risk.

Another objective of the present invention is to provide a kit for digitizing nutritional status, muscle turnover, and risk assessment, which comprises: Histidine, Leucine, Ornithine and Phenylalanine.

In one embodiment of the present invention, the Formula 4 further determines the level of muscle lysis; the Formula 3 further determines liver metabolic function; the Formula 1 further determines muscle turnover; and Formula the 5 further determines metabolite loading to liver.

In one embodiment of the present invention, the biological sample of the step (a) is blood, plasma, serum, red blood cells or urine.

In one embodiment of the present invention, the subject of the step (a) is a patient suffering from a disease or a healthy normal person.

In one embodiment of the present invention, the disease is aging, cancer, chronic diseases, severe diseases and cardiovascular diseases; and the chronic disease is chronic obstructive pulmonary disease (COPD), end stage of renal disease (ESRD) or chronic kidney disease (CKD).

In one embodiment of the present invention, the detecting method of the step (a) is a mass spectrometry (MS), liquid chromatograph (LC), micro-scale capillary electrophoresis or high performance capillary electrophoresis.

In one embodiment of the present invention, adjusting the subject's nutrition intervention and lifestyle according to result of the step (c).

In one embodiment of the present invention, the step (a) to the step (c) are done again after adjusting the nutrition intervention and lifestyle.

In one embodiment of the present invention, the step (a) further comprises to measure body fat, muscle weight, body water weight, body weight, daily dietary water intake of the subject.

In one embodiment of the present invention, the risk assessment comprises to assess the risk of death or hospitalization due to condition worsening.

In one embodiment of the present invention, the values obtained from Formulas 1 to 5 calculated based on genders are compared with the reference value at x-axis and y-axis of the map of digitizing nutritional status, muscle turnover, and risk assessment.

Accordingly, the present invention provides a method of digitizing nutritional status, muscle turnover, and risk assessment using the formulas containing four amino acids; it can be scientific to evaluate improvement or worsening of disease status based on digitizing. The method is an advanced nutrition assessment to guide new nutrition intervention and lifestyle adjustment for patients or healthy individuals, which can improve quality of life, improve bodily function, assist muscle growth, and reduce the occurrence of adverse events. In addition, the patients assessed by the method of the present invention are safer and have better treatment compared to those without assessed by the method of the present invention. The method of digitizing nutritional status, muscle turnover, and risk assessment is expected to create a personalized nutrition intervention, which achieves the purpose of precise medicine and improves the quality of medical care.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 shows a flowchart of the method of digitizing nutritional status, muscle turnover, and risk assessment of the present invention.

FIG. 2 is the map of the method of digitizing nutritional status, muscle turnover, and risk assessment. X-axis is nutritional status; Y-axis is risk assessment (from A0 to A8 or from A0 to A-5 represents higher risks, A0 represents lowest-risk, A8 and A-5 represents highest-risk).

FIG. 3 shows the event rate in the following six months in patients with a variety of diseases assessed by the method of digitizing nutritional status, muscle turnover, and risk assessment of the present invention. The event is defined as death or hospitalization due to condition worsening.

FIGS. 4A to 4C show a decision-making tree of risk assessment model I in male.

FIGS. 5A to 5E show a decision-making tree of risk assessment model I in female.

FIG. 6 shows that a very complicated patient significantly improved from poor to good status in response to appropriate nutritional intervention guided by the method of digitizing nutritional status, muscle turnover, and risk assessment.

### DETAILED DESCRIPTION OF THE PREFERRED EMBODIMENT

The present invention provides a method of digitizing nutritional status, muscle turnover, and risk assessment using the formulas containing four amino acids, which are Histidine, Leucine, Ornithine and Phenylalanine. The method is an advanced nutrition assessment to guide new nutrition interventions for patients or healthy individuals. It can innovate upon new designs of nutritional supplements and provide objective parameters based on digitalizing nutritional assessments to realize the effect of nutrition intervention and solve subject's problems. Thus, it can give better life quality and advanced medical care, improve quality of life, improve bodily functions and assist muscle growth. The method can also assess patient's disease state, provide digitalized staging of diseases and clarify the disease state of patient to plan for appropriate nutrition interventions accordingly and to lead to better rehabilitation outcomes under the guidance of the digitalized nutrition assessment.

As used herein, Numerical quantities given herein are approximate, meaning that can vary within a range of ±15%, preferably within ±10%, and most preferably within ±5%.

### EXAMPLE 1

The method of digitizing nutritional status, muscle turnover, and risk assessment

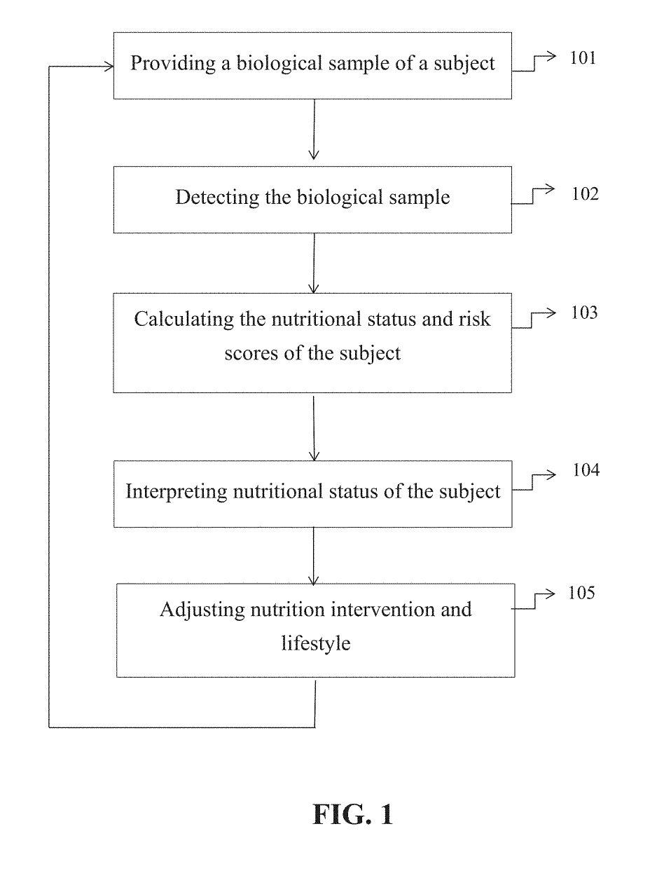

FIG. 1 shows the flowchart of the method of digitizing nutritional status, muscle turnover, and risk assessment of the present invention, comprising: Step 101: providing a biological sample of a subject, Step 102: detecting the biological sample; Step 103: calculating the nutritional status and risk scores of the subject; Step 104: interpreting nutritional status of the subject; and Step 105: adjusting nutrition intervention and lifestyle; reassessing the method of digitizing nutritional status, muscle turnover, and risk assessment of the present invention.

### 1.1 Providing a Biological Sample of a Subject

In the present invention, obtaining the biological sample from a subject, comprising: blood, plasma, serum, red blood cells or urine. In one embodiment of the present invention, the biological sample is air dried blood spot sample, or, the biological sample can be plasma, serum and red blood cells obtained after centrifugation for detection.

### 1.2 Detecting the Biological Sample

As an example for plasma, the present invention analyzes plasma using two methods, such as time-of-flight mass spectrometry (TOF MS) and Ultra performance liquid chromatograph (UPLC), or using additional methods, such as mass spectrometry and liquid chromatograph.

(1) Time-of-flight mass spectrometry (TOM MS): in the present invention, the metabolites were quantified by following procedures. To 50-μl plasma, 200 μl acetonitrile (ACN) was added. The mixture was vortexed for 30 s, sonicated for 15 min, and centrifuged at 10,000×g for 25 min The supernatant was collected into a separate glass tube. The pellets were re-extracted with 200 μl 50% methanol. The aqueous methanolic supernatant and acetonitrile supernatant were pooled and dried in a nitrogen evaporator. The residues were saved and stored at −80° C. For metabolomics analysis, the residues were suspended in 100 μL of 95:5 water/acetonitrile and centrifuged at 14,000×g for 5 min The clear supernatant was collected for Liquid chromatography-mass spectrometry (LC-MC) analysis.

Liquid chromatographic separation was achieved on a 100 mm×2.1 mm Acquity 1.7-μL C8 column (WATERS CORP., USA) using a ACQUITY™ UPLC system (WATERS CORP., USA). The column was maintained at 45° C., and at a flow rate of 1.5 mL/min Samples were eluted from LC column using a linear gradient: 0-2.5 min: 1-48% B; 2.5-3 min: 48-98% B; 3-4.2 min: 98% B; 4.3-6 min: 1% B for re-equilibration. The mobile phases were 0.1% formic acid (solvent A) in water and 0.1% formic acid in acetonitrile (solvent B).

The eluent was introduced into the TOF MS system (SYNAPT G1 high-definition mass spectrometer, WATERS CORP., USA) and operated in an ESI-positive ion mode. The conditions were as follows: desolation gas was set to 700 l/h at a temperature of 300° C., cone gas set to 25 l/h, and source temperature set at 80° C. The capillary voltage and cone voltage were set to 3,000 V and 35 V, respectively. The MCP detector voltage was set to 1,650 V. The data acquisition rate was set at 0.1 s with a 0.02 s inter scan delay. The data were collected in centroid mode from 20 to 990 m/z. For accurate mass acquisition, a lock-mass of sulfadimethoxine at a concentration of 60 ng/mL and a flow rate of 6 l/min (an [M+H]⁺ ion at 311.0814 Da in ESI-positive mode).

(2) Ultra performance liquid chromatograph (UPLC): due to the strong polar of amino acid, a UPLC protocol for the determination of amino acids after pre-column derivatization with 6-aminoquinolyl-N-hydroxysuccinimidyl carbamate (AQC) was used to increase a retention time in reversed phase liquid chromatograph. Applying a simple and easy to prepare binary eluent system, the 4 amino acids were separated in about 7 min with a total runtime of 10.5 min until the next injection. Since AQC amino acid derivatives were primarily designed to be used with fluorescence detection but facilitate UV detection as well. 6-aminoquinoline, the hydrolysis by-product of derivatization which implies similar absorbance as amino acid derivatives, was effectively separated prior to the polar amino acids, hence no interferences in UV detection were observed. For UV detection at 254 nm, all amino acids exhibited a quite similar response, whereas the respective fluorescence yield at 395 nm emission (excitation at 254 nm) indicated significant dependencies from the applied conditions mainly affected by aqueous quenching. Regarding the possible sensitivity compared to UV, fluorescence detection proved to be superior with detection limits ranging down to the low fmol level.

### 1.3 Calculating the Nutritional Status and Risk Scores of the Subject

In the present invention, the four amino acid (Histidine, Leucine, Ornithine, Phenylalanine) levels were first measured in 40 normal male and 40 normal female subjects. The mean, standard deviation, and 95% confidence interval of the four amino acids, muscle turnover, muscle lysis [corrected phenylalanine level (Pc)], liver metabolic function [corrected ornithine level (Oc)] and metabolite loading to liver.

Calculation of these parameters and clinical follow-up were conducted in 212 patients. The 5 parameters (muscle turnover, digitized nutritional status and risk scores, liver metabolic function, and metabolite loading to liver) were calculated by the formulas, all formulas can be modified according to requirement as follows:
(1) Formula 1 (muscle metabolism turnover): Histidine level/Phenylalanine level;
(2) Formula 2 (digitized nutritional status score): (−19.265 to −15.763)×(the value of Formula 1)+(0.059 to 0.073)×Ornithine level+(18.776 to 22.948);
(3) Formula 3 (liver metabolic function): the value of Formula 3 calculated based on gender is corrected Ornithine level (Oc):
   Male:
if Leucine level≤[(mean of Leucine level in normal male blood)−(standard deviation of Leucine level in normal male blood)],
   Oc=[Ornithine level×[(mean of Leucine level in normal male blood)−(standard deviation of Leucine level in normal male blood)]]/Leucine level;
if Leucine level>[(mean of Leucine level in normal male blood)−(standard deviation of Leucine level in normal male blood)],
   Oc=Ornithine level;
   Female:
if Leucine≤[(mean of Leucine level in normal female blood)−(standard deviation of Leucine level in normal female blood)],
   Oc=[Ornithine level×[(mean of Leucine level in normal female blood)−(standard deviation of Leucine level in normal female blood)]]/Leucine level;
if Leucine level>[(mean of Leucine level in normal female blood)−(standard deviation of Leucine level in normal female blood)],
   Oc=Ornithine level;
(4) Formula 4 (muscle lysis): the value of Formula 4 calculated based on gender is corrected Phenylalanine level (Pc):
   Male:
if Leucine level≤[(mean of Leucine level in normal male blood)−(standard deviation of Leucine level in normal male blood)],
   Pc=[Phenylalanine level×[(mean of Leucine level in normal male blood)−(standard deviation of Leucine level in normal male blood)]]/Leucine level;
if Leucine level>[(mean of Leucine level in normal male blood)−(standard deviation of Leucine level in normal male blood)],
   Pc=Phenylalanine level;
   Female:
if Leucine level≤[(mean of Leucine level in normal female blood)−(standard deviation of Leucine level in normal female blood)],
Pc=[Phenylalanine level×[(mean of Leucine level in normal female blood)−(standard deviation of Leucine level in normal female blood)]]/Leucine level;
if Leucine level>[(mean of Leucine level in normal female blood)−(standard deviation of Leucine level in normal female blood)],
   Pc=Phenylalanine level; and
(5) Formula 5 (metabolite loading to liver)=−[(−1.414 to −1.157)×Ornithine level/(Leucine level+Histidine level−Phenylalanine level)+(0.0063 to 0.0077)×(the value of Formula 3)]×(9 to 11)

The patients included those with diabetes, hypertension, arrhythmia, chronic obstructive pulmonary disease (COPD), chronic kidney disease (CKD) and cardiovascular diseases. The average age of them is 60.7 (as shown in Table 1). On one embodiment of the present invention, Formula 2 may be −17.514×(the value of Formula 1)+0.66×Ornithine level+20.862; Formula 5 may be −[(−1.285)×Ornithine level/(Leucine level+Histidine level−Phenylalanine level)+0.007×(the value of Formula 3)]×10. All patents are subjected to the method of digitizing nutritional status, muscle turnover, and risk assessment of the present invention. The location of risk area was calculated. The rate of event at each risk area was assessed during a follow-up period of six months. The event is defined as death or hospitalization due to condition worsening.

TABLE 1
The clinical characteristics of the patients

| | All (n = 212) | |
|---|---|---|
| Age (years) | 60.7 ± 12.9 | |
| Male (%) | 146 | (68.9) |
| Blood pressure (mm Hg) | | |
| Systolic | 122.2 ± 17.6 | |
| Diastolic | 74.1 ± 12.1 | |
| Heart rate (beats/min) | 77.9 ± 11.2 | |
| Disease | | |
| Cardiovascular disease (%) | 185 | (87.3) |
| Diabetes (%) | 90 | (42.5) |
| Hypertension (%) | 142 | (67.0) |
| Atrial fibrillation (%) | 67 | (31.6) |
| COPD (%) | 27 | (12.7) |
| Chronic kidney disease (%) | 41 | (19.3) |
| Body mass index (kg/m²) | 25.2 ± 5.4 | |
| Laboratory data | | |

| | |
|---|---|
| Albumin (g/dl) | 3.6 ± 0.5 |
| eGFR (ml/min/1.73 m²) | 71.1 ± 23.5 |

COPD: chronic obstructive pulmonary disease;
eGFR: the estimated glomerular filtration rate

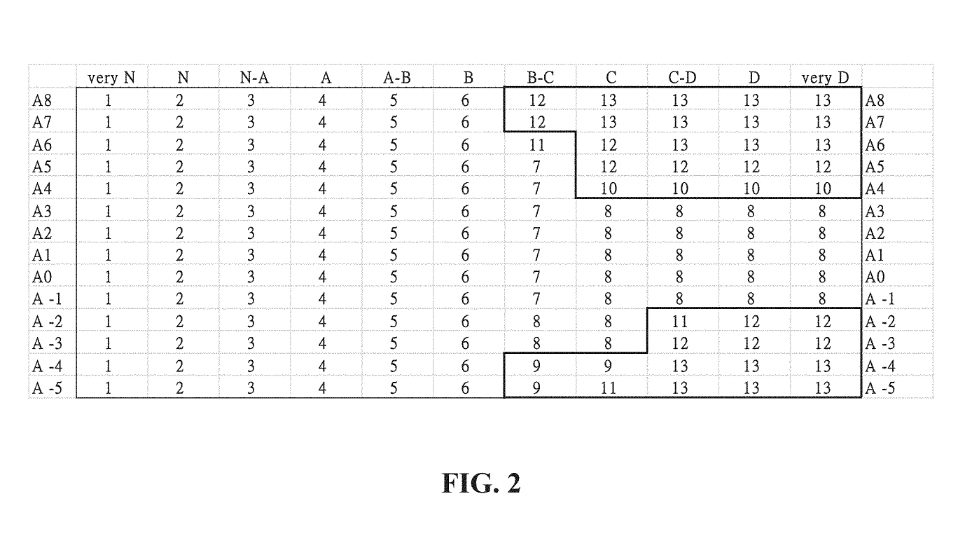

First, the severity of nutritional status of each patent is digitized on a scale from normal to extremely severe using Formulas 1 and 2. The severity on the scale is labeled as N, A, B, C, D (x-axis of FIG. 2), which are further subdivided into very N (very normal), N (normal), N-A (normal to early abnormal), A (early abnormal), A-B (early abnormal to significantly abnormal but no symptom), B (significantly abnormal but no symptom), B-C (significantly abnormal but no symptom to very abnormal), C (very abnormal), C-D (very abnormal to extremely abnormal), D (extremely abnormal), very (very extremely abnormal).

First, the numerical range of normal person (N) is calculated by normal person, and then the numerical range of A, B, C and D are separately defined based on female and male.

Female:

the lower limit of 95% confidence interval of N≤N≤the upper limit of 95% confidence interval of N;

the lower limit of 95% confidence interval of N+4.42≤A≤the lower limit of 95% confidence interval of N+5.96;

the lower limit of 95% confidence interval of N+7.06≤B≤the lower limit of 95% confidence interval of N+9.01;

the lower limit of 95% confidence interval of N+11.75≤C≤the lower limit of 95% confidence interval of N+13.52;

the lower limit of 95% confidence interval of N+18.07≤D≤the lower limit of 95% confidence interval of N+23.66.

Male:

the lower limit of 95% confidence interval of N≤N≤the upper limit of 95% confidence interval of N;

the lower limit of 95% confidence interval of N+3.3≤A≤the lower limit of 95% confidence interval of N+4.46;

the lower limit of 95% confidence interval of N+6.96≤B≤the lower limit of 95% confidence interval of N+7.64;

the lower limit of 95% confidence interval of N+11.36≤C≤the lower limit of 95% confidence interval of N+12.35;

the lower limit of 95% confidence interval of N+18.04≤D≤the lower limit of 95% confidence interval of N+20.77.

The numerical range of very N, N-A, A-B, B-C, C-D, very D are defined according to the numerical range of N, A, B, C, D. In one embodiment, the numerical range of each scale is show in Table 2.

TABLE 2
The nutritional status score of each scale

| | Female | Male |
|---|---|---|
| very D | >23.15 | >21.33 |
| D | ≥17.56 | ≥18.6 |
| C-D | >13.01 | >12.91 |
| C | ≥11.24 | ≥11.92 |
| B-C | >8.5 | >8.2 |
| B | ≥6.55 | ≥7.52 |
| A-B | >5.45 | >5.02 |
| A | ≥3.91 | ≥3.86 |
| N-A | >1.89 | >2.95 |
| N | ≥−0.51 | ≥0.56 |
| very N | <−0.51 | <0.56 |

Then, the risk location at y-axis of the map of the method of digitizing nutritional status, muscle turnover, and risk assessment as shown in FIG. 2 is determined by the value of Formulas 3 to 5, and is assessed as the risk level (from A0 to A8 or from A0 to A-5 represents a higher risk; A0 represents lowest-risk, A8 and A-5 represent highest-risk) and risk score. A risk score of 9 or over represents that the risk of hospitalization or death in the following six months is higher than 50%. The higher the risk score, the greater the risk.

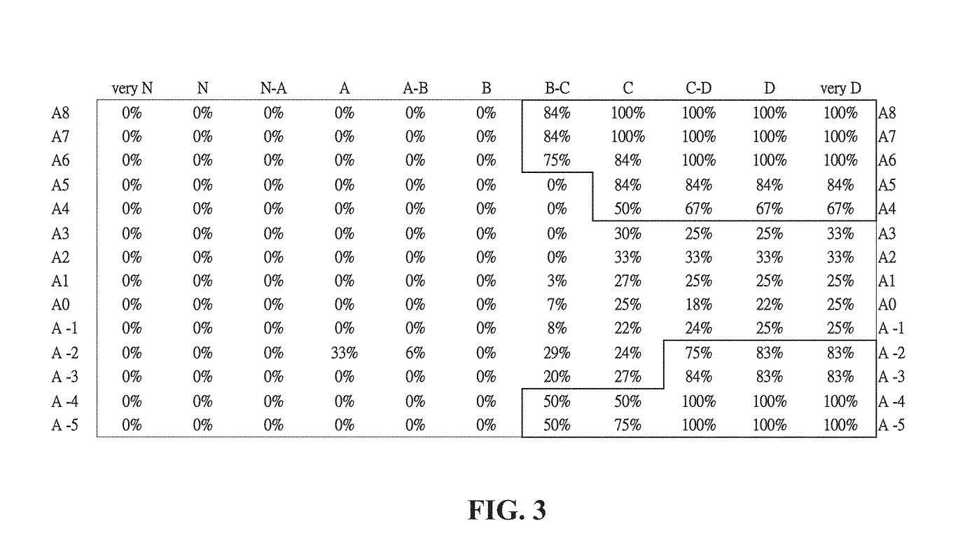

During the period of following up the 212 patients for 6 months, there were 82 patients (38.7%) hospitalized due to condition worsening/or death due to condition worsening. FIG. 3 shows the predictive value of the method of digitizing nutritional status, muscle turnover, and risk assessment of the present invention on the event rate in the following six months in patients with different diseases. The event is defined as death or hospitalization due to condition worsening. The method of the present invention was assessed to see whether it could predict events in the following six months. The event occurred in 82 patients (38.7%).

The risk assessment model is calculated separately for male and female. There are two independent calculation methods, namely, model I and model II. The final risk assessment is determined by the cooperation mode of model I and model II. The details are as follows:

Male:

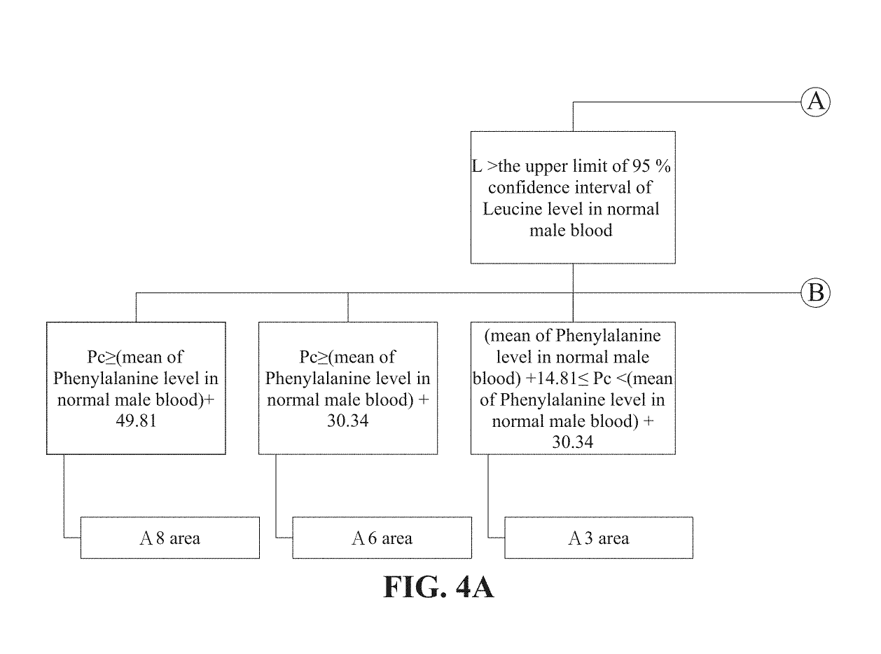

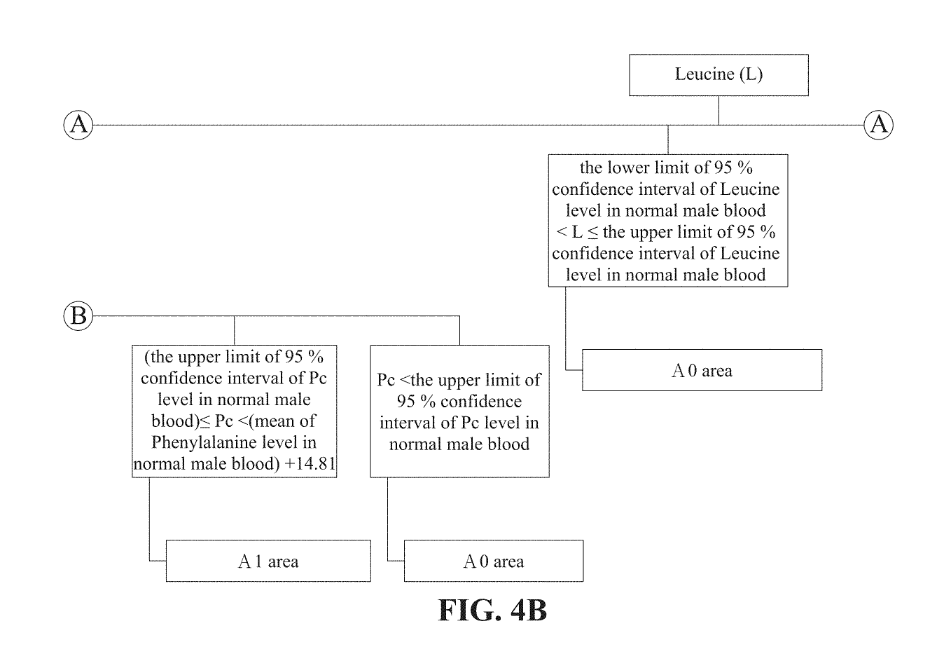

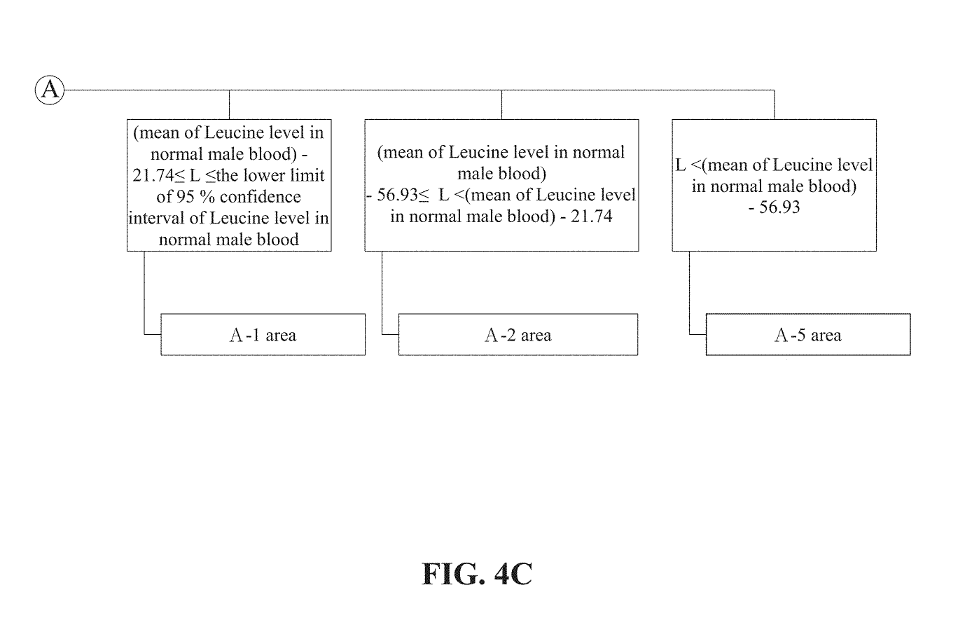

(1) Risk assessment model I: the risk level is determined in accordance with the decision-making tree in FIGS. 4A to 4C.

(2) Risk assessment model II: in the case of Oc>the upper limit of 95% confidence interval of Oc level in normal male blood, if the value of metabolite loading to liver (Formula 5)≤[(mean of the value of metabolite loading to liver of normal male)+1.9795], then the risk increases, which is at A4 or A-3 of the risk level (if Leucine level≤mean of Leucine level in normal male blood, the risk level is A4; if Leucine level<mean of Leucine level in normal male blood, the risk level is A-3); if the value of metabolite loading to liver (Formula 5)≤[(mean of the value of metabolite loading to liver of normal male)+4.7792], then the risk increases, which is at A7 or A-4 of the risk level (if Leucine level>mean of Leucine level in normal male blood, the risk level is A7; if Leucine level<mean of Leucine level in normal male blood, the risk level is A-4).

(3) In the cooperation mode of model I and model II, the final risk level is determined by the higher risk level of either of the two models. For example: if the risk level assessed by model I is A3, and the risk level assessed by model II is A7, then the final risk level is A7.

Female:

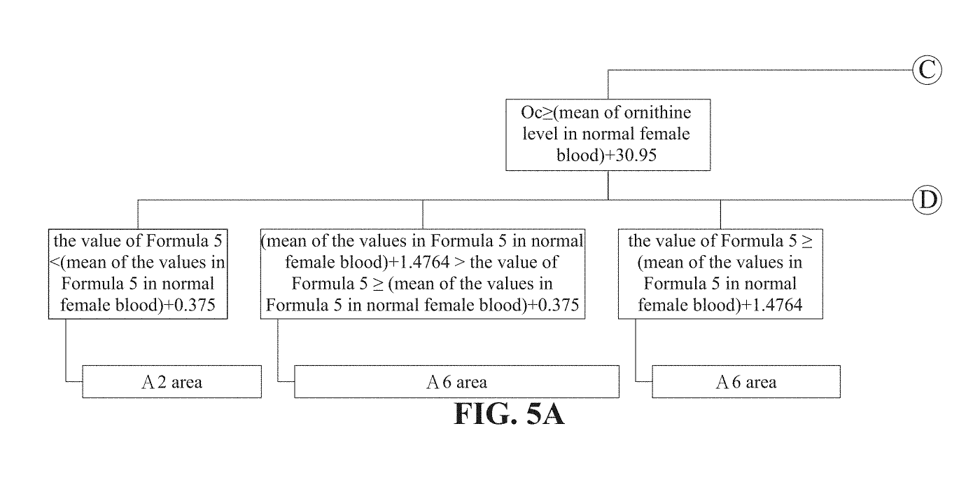

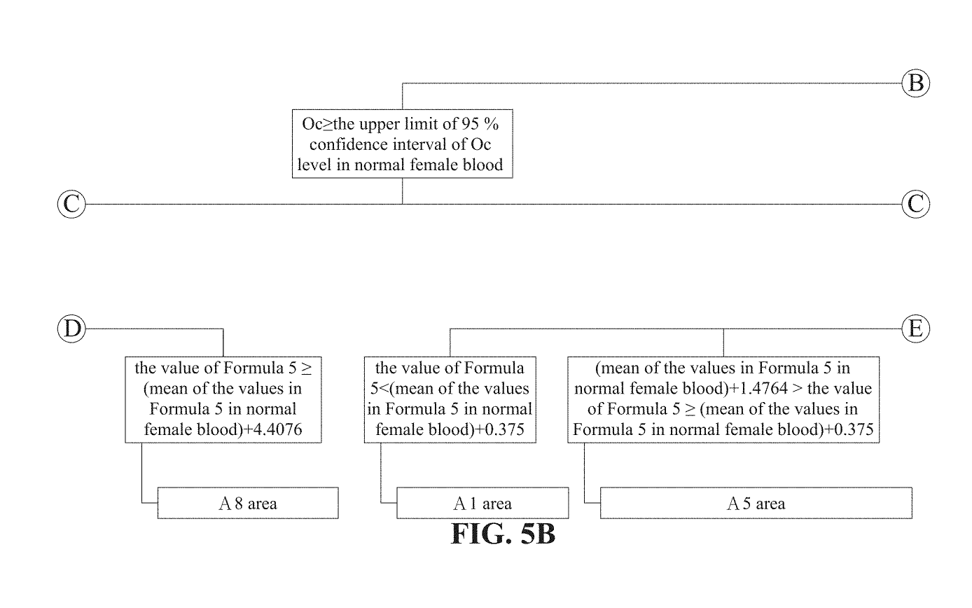

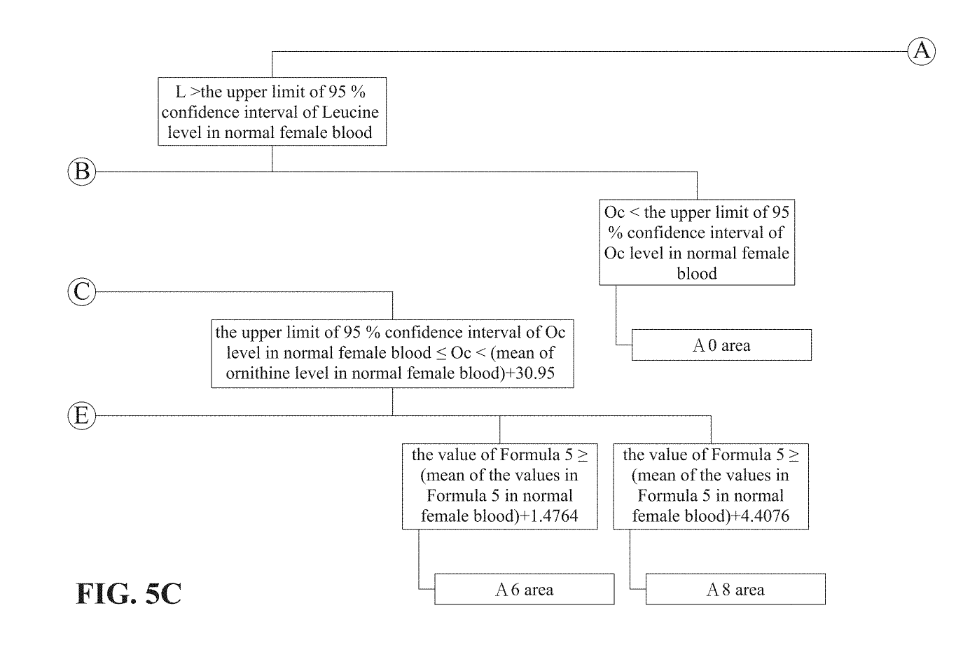

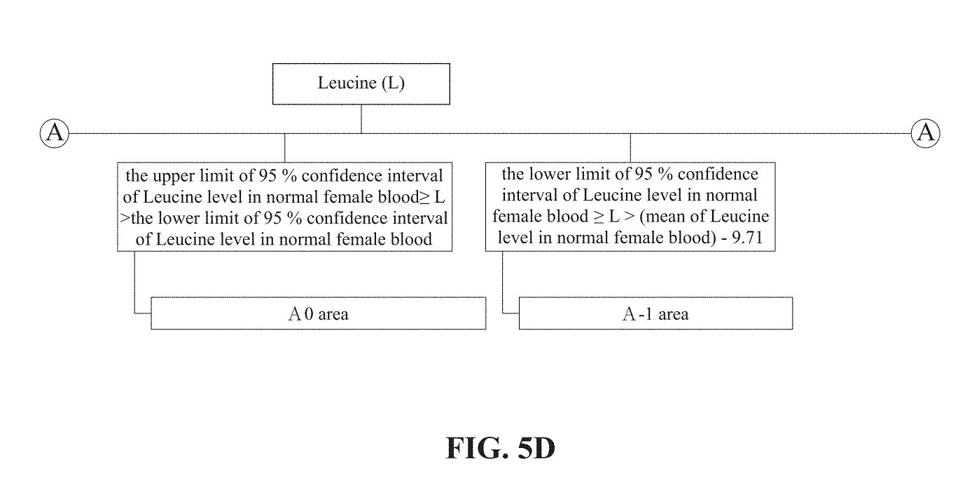

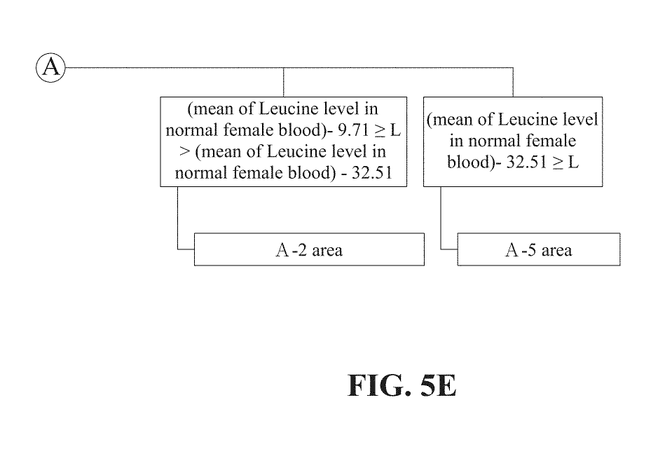

(1) Risk assessment model I: the risk level is determined in accordance with the decision-making tree in FIGS. 5A to 5E.

(2) Risk assessment model II: in the case of Oc>the upper limit of 95% confidence interval of Oc level in normal female blood, if the value of metabolite loading to liver (Formula 5)≤[(mean of the value of metabolite loading to liver of normal female)+1.4764], then the risk increases, which is at A4 or A-3 of the risk level (if Leucine level ≤mean of Leucine level in normal female blood, the risk level is A4; if Leucine level<mean of Leucine level in normal female blood, the risk level is A-3); if the value of metabolite loading to liver (Formula 5)≤[(mean of the value of metabolite loading to liver of normal female)+4.4076], then the risk increases, which is at A7 or A-4 of the risk level (if Leucine level>mean of Leucine level in normal female blood, the risk level is A7; if Leucine level<mean of Leucine level in normal female blood, the risk level is A-4).

(3) In the cooperation mode of model I and model II, the final risk level is determined by the higher risk level of either of the two models. For example: if the risk level assessed by model I is A3, and the risk level assessed by model II is A7, then the final risk level is A7.

For the severity of muscle lysis (Pc), the severity is defined as:

Male:

Slightly high: the severity of muscle lysis (Pc) is between the upper limit of 95% confidence interval of Pc level in normal male blood and the upper limit of 95% confidence interval of Pc level in normal male blood+24.79;

High: the severity of muscle lysis (Pc) is between the upper limit of 95% confidence interval of Pc level in normal male blood+24.79 and the upper limit of 95% confidence interval of Pc level in normal male blood+44.79; and

Very high: the severity of muscle lysis (Pc) >the upper limit of 95% confidence interval of Pc level in normal male blood+44.79.

Female:

Slightly high: the severity of muscle lysis (Pc) is between the upper limit of 95% confidence interval of Pc level in normal female blood and the upper limit of 95% confidence interval of Pc level in normal female blood+23.27;

High: the severity of muscle lysis (Pc) is between the upper limit of 95% confidence interval of Pc level in normal female blood+23.27 and the upper limit of 95% confidence interval of Pc level in normal female blood+43.27; and

Very high: the severity of muscle lysis (Pc)>the upper limit of 95% confidence interval of Pc level in normal female blood+43.27.

For the severity of liver metabolic function (Oc), the severity is defined as:

Male:

Slightly high: the severity of liver metabolic function (Oc) is between the upper limit of 95% confidence interval of Oc level in normal male blood and the upper limit of 95% confidence interval of Oc level in normal male blood+25;

High: the severity of liver metabolic function (Oc) is between the upper limit of 95% confidence interval of Oc level in normal male blood+25 and the upper limit of 95% confidence interval of Oc level in normal male blood+45; and

Very high: the severity of liver metabolic function (Oc) >the upper limit of 95% confidence interval of Oc level in normal male blood+45.

Female:

Slightly high: the severity of liver metabolic function (Oc) is between the upper limit of 95% confidence interval of Oc level in normal female blood and the upper limit of 95% confidence interval of Oc level in normal female blood+24.51;

High: the severity of liver metabolic function (Oc) is between the upper limit of 95% confidence interval of Oc level in normal female blood+24.51 and the upper limit of 95% confidence interval of Oc level in normal female blood+44.51; and

Very high: the severity of liver metabolic function (Oc)>the upper limit of 95% confidence interval of Oc level in normal female blood+44.51.

The abnormality and abnormal level of muscle turnover and metabolite loading to liver are all determined by mean and 95% confidence interval of muscle turnover and metabolite loading to liver in normal male and female.

The abnormality and abnormal level of amino acid level in blood are all determined by mean and 95% confidence interval of amino acid levels in blood of normal male and female.

### 1.4 Interpreting Nutritional Status of the Subject

The risk level and risk score are interpreted by the method of the present invention to provide personalized nutritional intervention and life style adjustment, comprising: nutritional status and risk assessment, the severity of muscle lysis, amino acid level in blood, liver metabolic function, muscle turnover and metabolite loading to liver.

### EXAMPLE 2

### The Clinical Application of the Method of Digitizing Nutritional Status, Muscle Turnover, and Risk Assessment

The method of digitizing nutritional status, muscle turnover, and risk assessment can be used to determine the digitized stages of various diseases, such as cardiovascular disease, aging, cancer, chronic obstructive pulmonary disease (COPD), end stage of renal disease (ESRD), chronic kidney disease (CKD), to recovery from a critical illness (especially patients with complex diseases) and to monitor improvement or worsening of diseases, to recovery after surgery and heal wound, to assess nutritional status, the effect of nutrition intervention and prognosis in patients with critical illness, to assist metabolic balance in patients with critical illness, to improve quality of life, bodily functions and muscle growth; to fine-tune nutrition intervention avoiding over-supplementation and improving disease prognosis, to determine the timing of rehabilitation and to provide nutrition intervention leading to the best rehabilitation results, and to conduct health check on healthy individuals assessing nutritional status and predicting diabetes.

### 2.1 The Method is Applied to a Patient Suffering from Complex Diseases in an Intensive Care Unit

A 76 y/o female was admitted to Intensive Care Unit (ICU) due to acute respiratory failure. She had diabetes mellitus, hypertension, atrial fibrillation, chronic kidney disease. Her heart function was normal and albumin level was normal (=3.7 g/dl).

According to the first result of the method of digitizing nutritional status, muscle turnover, and risk assessment: nutritional status was C-D, risk level was A8 (risk area); severe muscle lysis, very high blood amino acid levels, impaired liver metabolic function, and inadequate amino acid supply along with inadequate muscle turnover and inadequate muscle growth. These findings suggested (1) risk status; (2) patient's liver cannot deal with the metabolic waste produced from amino acid metabolism (3) need to support calories by carbohydrates rather than protein products to decrease muscle breakdown; (4) body in extreme breakdown state, full of amino acids, but the body was not able to use these as energy; (5) cannot supply patient with large amount of amino acids, because the patient cannot use these amino acids appropriately and will produce a lot of waste.

Based on these guides, the patient was treated based on precision medicine. She was transferred out of the ICU 7 days later and then taking rehabilitation. The measurement of digitizing nutritional status, muscle turnover, and risk assessment was reassessed 10 days later and showed: status A, located at A2 (safe area); no muscle lysis, very low amino acid level, normal liver metabolic function, normal muscle turnover. These findings suggested (1) her condition improved a lot; (2) no more muscle lysis; (3) remarkable deficiency in amino acid amount; (4) it is safe to supply amino acids; (5) need to supply more amino acids, otherwise, tissue repair will be affected. After personalized nutrition intervention, a status of muscle growing was found by body composition scale (OSERIO, Taiwan). The body muscle weight at the time of reassessing the measurement of digitizing nutritional status, muscle turnover, and risk assessment was 25 kg. After one week, the body muscle weight increased up to 26.2 kg. The 6-min walking distance increased from 49 meters to 152 meters.

### 2.2 The Method is Applied to a Patient with COPD

A 71 y/o male of COPD, with normal cardiac and kidney function. Albumin level was 4.5 g/dl.

According to the result of the method of digitizing nutritional status, muscle turnover, and risk assessment: nutritional status was D, risk level was A7 (risk area); very high muscle lysis, very low blood amino acid level, severely impaired liver metabolic function, increased but normal muscle turnover. These findings suggested (1) risk status; (2) need some ways to decrease muscle lysis (such as supplying adequate calories by carbohydrates); (3) need to decrease the situation of using amino acids as energy source so as to decrease the loading of liver metabolism; (4) supply limited amount of amino acids; otherwise, they will turn into loadings on dysfunctional liver; (5) need to train skeletal muscle to turn amino acids from “being used as energy” to “being used for muscle synthesis”; (6) of course, need to treat the underlying disease.

### 2.3 The Method is Applied to a Critically Ill Patient in an Intensive Care Unit

A 52 y/o male was admitted to an Intensive Care Unit due to septic shock. His blood albumin level was 2.7 g/dl.

According to the result of the method of digitizing nutritional status, muscle turnover, and risk assessment: nutritional status was very D, risk level was A7 (risk area); very high muscle lysis, low blood amino acid level, normal liver metabolic function, inadequate muscle turnover due to remarkably inadequate nutritional support (amino acids) for this. These findings suggested (1) risk status; (2) need some ways to decrease muscle lysis (such as supplying adequate calories by nutrition including carbohydrate, fat and limited amount of high-quality amino acids, decreasing steroid use) (decrease muscle lysis, decrease the situation of using amino acids as energy source to decrease the loading of liver metabolism); (3) supply limited amount of high-quality amino acids (especially more Histidine, Branch Chain Amino Acids, but less phenylalanine); (4) of course, need to treat the underlying disease.

However, without using the method of digitizing nutritional status, muscle turnover, and risk assessment, this patient was given with high dose of albumin Intravenous (IV) infusion for a few days. Then, the method of digitizing nutritional status, muscle turnover, and risk assessment was reassessed and showed “status very D, located at A8”, suggesting worsening. The patient was expired 19 days later.

### 2.4 The Method is Applied to a Cancer Patient Undergoing Chemotherapy

A 43 y/o male was a case of cancer, under chemotherapy treatment (oncology).

According to the result of the method of digitizing nutritional status, muscle turnover, and risk assessment: nutritional status was D, risk level was A7 (risk area); slight muscle lysis, very low blood amino acid level, slight liver metabolic dysfunction, inadequate muscle turnover due to remarkably inadequate nutritional support (amino acids) for this. These findings suggested (1) risk status; (2) need some ways to decrease muscle lysis (such as supplying adequate calories by carbohydrate and fat) (supplying more high-quality amino acids such as more histidine, Branch Chain Amino Acids, but less phenylalanine); (3) of course, need to treat the underlying disease.

To follow up the measurement of digitizing nutritional status, muscle turnover, and risk assessment was needed to focus on whether the supply of amino acids caused increased liver metabolic loading. After this interventional strategy, the body muscle mass of this patient increased from 27.3 kg to 29.2 kg after one month.

### 2.5 The Method is Applied to a Patient Suffering from Complex Diseases of Cardiovascular Disease and Chronic Kidney Disease

A 35 y/o male, was a case of cardiovascular disease and chronic kidney disease. His blood albumin level was 2.5 g/dl.

According to the result of the method of digitizing nutritional status, muscle turnover, and risk assessment: nutritional status was D, risk level was A8 (risk area); slight muscle lysis, very low blood amino acid level, slight liver metabolic dysfunction, inadequate muscle turnover due to remarkably inadequate nutritional support (amino acids) for this, and very high loading of metabolites to liver. These findings suggested (1) high risk status; (2) need some ways to decrease muscle lysis (such as supplying adequate calories by carbohydrate and fat, but not supplying a large amount of protein); (3) supply limited amount of amino acids such as more histidine, branch chain amino acids, but less phenylalanine. However, the liver metabolic function and the metabolite loading to liver should be monitored. The supply of amino acids should not increase these parameters; (4) under adequate calorie supply, may do some limited amount of exercise to decrease muscle lysis, and guide the body to use amino acids for muscle synthesis instead of for energy production; (5) of course, needing to treat the underlying disease.

However, without using the method of digitizing nutritional status, muscle turnover, and risk assessment, this patient was given with lots amount of protein to increase blood albumin level in a few days without knowing the change in the liver metabolism function and the metabolite loading to liver. The patient died in one week.

### 2.6 The Method is Applied to a Patient Undergoing Kidney Dialysis, Combined with Respiratory Failure and Cardiovascular Disease

A 72 y/o male was a case of end stage renal disease, respiratory failure, cardiovascular disease, and cachexia.

According to the result of the method of digitizing nutritional status, muscle turnover, and risk assessment: nutritional status was very D, risk level was A7 (risk area); severe muscle lysis, slightly low amino acid level, normal liver metabolic function, inadequate muscle turnover due to severe muscle lysis, and very high metabolite loading to liver. These findings suggested (1) high risk status; (2) need some ways to decrease muscle lysis (such as supplying adequate calories by balanced nutrition, including carbohydrate, protein and fat, but less phenylalanine); (3) need to follow up whether the nutritional intervention decrease muscle lysis severity; (4) very high loading of metabolites to liver due to severe muscle lysis (5) of course, need to treat the underlying disease.

The patient was given a general nutritional intervention without using the method of digitizing nutritional status, muscle turnover, and risk assessment. The method of digitizing nutritional status, muscle turnover, and risk assessment is assessed 7 days later: nutritional status was very D, risk level was A8 (risk area); severe muscle lysis, very low blood amino acid level, normal liver metabolic function, and inadequate muscle turnover and severe muscle lysis due to inadequate nutritional supply (such as amino acids), and very high loading of metabolite to liver. These findings suggested (1) still at a high risk status, with worsening; (2) need some ways to decrease muscle lysis (such as supplying adequate calories by carbohydrate, fat, and more amino acids including more histidine, branch chain amino acids, but less phenylalanine); (3) need to follow up whether the nutritional intervention decreased muscle lysis severity; (4) need to follow up whether the nutritional intervention decreased very high loading of metabolites to liver due to severe muscle lysis; (5) of course, need to treat the underlying disease.

Without using the method of digitizing nutritional status, muscle turnover, and risk assessment, the response and adequacy of nutritional supply are very difficult to realize scientifically. This inadequacy leads to inefficient nutritional intervention.

### 2.7 The Method is Applied to an Elderly Patient Suffering from Sarcopenia

This patient was an 85 y/o female. She asked for help due to weakness related to aging and probably sarcopenia. Physicians told her that her problems were all related to aging. However, the result of the method of digitizing nutritional status, muscle turnover, and risk assessment revealed different findings as follows (although her complaints were the same, the findings in the metabolic panel were totally different).

According to the first result of the method of digitizing nutritional status, muscle turnover, and risk assessment: nutritional status was very D, risk level was A7 (risk area); moderate muscle lysis, very low blood amino acid level, severe liver metabolic dysfunction, inadequate muscle turnover due to very low amino acids level and muscle lysis, and high metabolite loading to liver. These findings suggested (1) she was at high risk status; (2) the patient used amino acids for energy production, which increased liver loading; (3) need some ways to decrease the situation of using amino acids as energy source, and to decrease the metabolite loading to liver (such as supplying adequate calories by carbohydrate); (4) need to supply limited amount of high-quality amino acids (including more histidine, branch chain amino acids, but less phenylalanine), however, could not increase the metabolite loading to liver; (5) need to follow up whether the nutritional intervention decreased muscle lysis severity and liver loading, but improved liver metabolism function, and increased blood amino acids level.

The method of digitizing nutritional status, muscle turnover, and risk assessment was reassessed 3 months later: nutritional status was C, risk level was A3 (safe area); severe muscle lysis, normal blood amino acid level, normal liver metabolic function, and inadequate muscle turnover due to severe muscle lysis. These findings suggested (1) her risk improved; (2) need some ways to decrease muscle lysis, and to decrease the situation of using amino acids as energy source (such as exercising or muscular training by rehabilitation course); (3) of course, need to treat the underlying disease. Based on this, rehabilitation training was performed for her. Gradually, skeletal muscle mass increased (skeletal muscle mass of the patient at the time of reassessing the method of digitizing nutritional status, muscle turnover, and risk assessment was 21 kg, after two weeks, skeletal muscle mass of the patient was increased to 22.3 kg, the 6-min walking distance was increased from 42 meters to 75 meters), which demonstrated the effect of the method of digitizing nutritional status, muscle turnover, and risk assessment for guided rehabilitation.

The method of present invention reiterates that although the clinical presentation are the same, the findings in the method of digitizing nutritional status, muscle turnover, and risk assessment may be totally different, which is related to personalized medicine.

### 2.8 The Method is Applied to an Aging Patient Suffering from Complex Diseases Including Chronic Lung Disease, Kidney Disease, Cardiovascular Disease, Cancer, Diabetes and Hypertension

A 75 y/o female had very complex diseases including aging, chronic obstructive pulmonary disease, chronic kidney disease, cardiovascular disease, cancer, diabetes mellitus and hypertension.

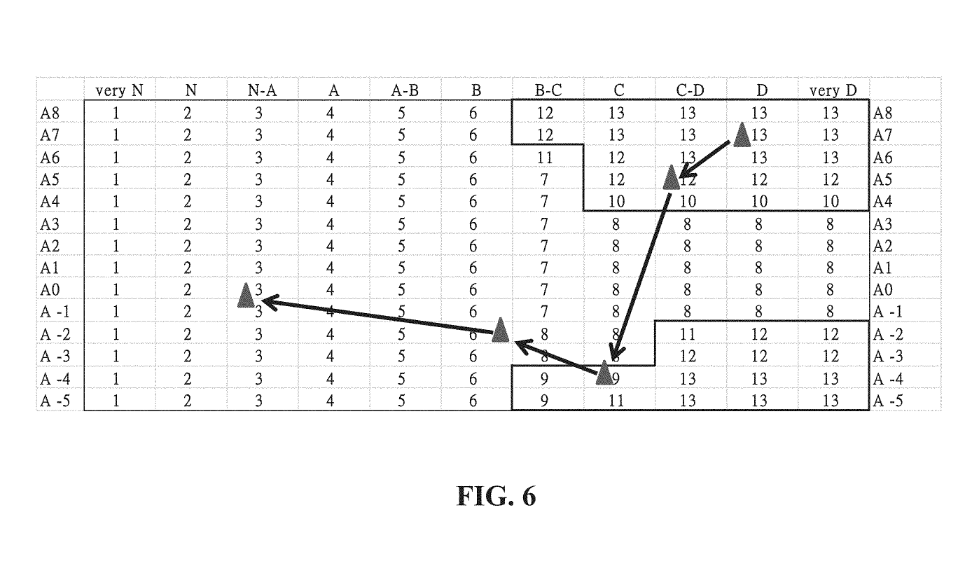

Using the method of digitizing nutritional status, muscle turnover, and risk assessment to evaluate the nutritional status of the patient and to guide the nutritional intervention. A series of risk scores at different time points are shown in FIG. 6. The scores begin at high risk status, but gradually improve. Finally, the score reaches the status of normal. The patient was stabilized at this time point. FIG. 6 shows a successful treatment story for a very complicated patient. Without the guide of the method of the present invention, the status of the patient could not be escalated safely with a good outcome.

### 2.9 The Method is Applied to Patients Suffering from Various Types of Chronic Diseases

102 patients were eligible to participate in the study, they must meet the following criteria: (1) age was over 20 years old; (2) creatinine level (kidney function)<2 g/dl; (3) having walking ability; (4) walking as a rehabilitation twice a day for 30 mins; (5) in addition to daily basic nutritional needs, daily supplying Histidine (1.0 g), Leucine (5.25 g), Isoleucine (1.2 g), Valine (2.25 g); (6) using body composition scale (OSERIO, Taiwan) to measure body weight, body fat, muscle and body water weight; (7) dietary advice and intervention by the same dietitian; (8) nutritional status at C.

According to the method of digitizing nutritional status, muscle turnover, and risk assessment, the patients were divided into two groups (as shown in Table 3), the risk levels of the patients in Group I were at A0, A1, A2, and A-1; the risk levels of the patients in Group II were at A-4, A-5, A5, and A6. The clinical presentation between the two groups was the same, such as baseline characteristics, body weight, muscle weight and 6-min walking distance, without statistically significant differences (Table 3 and Table 4). The difference between the two groups could only be distinguished by the method of digitizing nutritional status, muscle turnover, and risk assessment. The patients in Group I were all at lower risk as assessed by the method of digitizing nutritional status, muscle turnover, and risk assessment. The patients in Group II were all at higher risk as assessed by the method of digitizing nutritional status, muscle turnover, and risk assessment. The patients in the two groups were managed blindly by the same dietitian in the situation of without knowing the results of the method of digitizing nutritional status, muscle turnover, and risk assessment. They accepted the same amount of rehabilitation exercise, and were supplied the same amount of additional amino acids. As shown in Table 4, only the patients in the Group I showed a statistically significant improvement in muscle weight and 6-min walking distance after one month. These results validates that the method of digitizing nutritional status, muscle turnover, and risk assessment can effectively identify the patients who have a potential for muscle growth and better rehabilitation results, and thus improve their quality of life.

TABLE 3
The baseline characteristics of the low risk (Group I) and the high
risk (Group II) groups defined by the method of digitizing nutritional
status, muscle turnover, and risk assessment is applied to patients
suffering from various types of chronic diseases

| | Group I (44 patients) | | Group II (58 patients) | | P value |
|---|---|---|---|---|---|
| Age (years) | 60.8 ± 10.7 | | 62.9 ± 11.5 | | 0.363 |
| Male (%) | 38 | (86.4) | 49 | (84.5) | 1.000 |
| Blood pressure (mm Hg) | | | | | |
| Systolic | 123.9 ± 20.2 | | 125.2 ± 22.5 | | 0.763 |
| Diastolic | 70.1 ± 11.4 | | 70.4 ± 11.8 | | 0.899 |
| Heart rate, beats/min | 73.8 ± 12.7 | | 75.7 ± 16.0 | | 0.520 |
| Diseases | | | | | |
| Cardiovascular disease (%) | 100 | (100%) | 100 | (100) | 0.489 |
| Diabetes mellitus (%) | 9 | (20.5) | 16 | (27.6) | 0.489 |
| Hypertension (%) | 22 | (50.0) | 36 | (62.1) | 0.234 |
| Atrial fibrillation (%) | 5 | (11.4) | 13 | (22.4) | 0.193 |
| Chronic kidney disease (%) | 11 | (25) | 16 | (27.6) | 0.823 |
| COPD (%) | 4 | (9.3) | 6 | (10.3) | 1.000 |
| Body mass index (kg/m²) | 24.8 ± 3.4 | | 24.3 ± 3.4 | | 0.497 |
| Laboratory data | | | | | |

| | | | |
|---|---|---|---|
| Cholesterol (mg/dL) | 188.9 ± 46.7 | 182.9 ± 40.5 | 0.491 |
| Triglyceride (mg/dL) | 141.3 ± 104.2 | 145.5 ± 110.8 | 0.774 |
| Hemoglobin (g/dL) | 14.2 ± 1.9 | 13.7 ± 2.9 | 0.352 |
| Albumin (g/dl) | 4.3 ± 0.4 | 4.2 ± 0.5 | 0.470 |
| eGFR (ml/min/1.73 m²) | 67.3 ± 29.0 | 67.3 ± 21.2 | 0.998 |

COPD: chronic obstructive pulmonary disease;
eGFR: the estimated glomerular filtration rate

TABLE 4
The data of body weight, muscle weight, body fat weight, body water weight percentage
and 6-min walking distance of the low risk (Group I) and the high risk (Group II) groups
defined by the method of digitizing nutritional status, muscle turnover, and risk assessment
is applied to patients suffering from various types of chronic diseases

| | Group I (44 patients) | | Group II (58 patients) | | |
|---|---|---|---|---|---|
| | At beginning | After two weeks | At beginning | After two weeks | P value |
| nutrition status | C | | C | | |
| at beginning | | | | | |
| risk area | A0, A1, A2, A-1 | | A-4, A-5, A5, A6 | | |
| body weight (kg) | 67.66 ± 11.22 | 67.93 ± 11.57 | 66.06 ± 12.68 | 65.69 ± 12.7 | 0.43 |
| muscle weight (kg) | 26.59 ± 4.35 | 27.15 ± 4.56 | 26.51 ± 5.24 | 25.99 ± 5.13 | 0.01 |
| body fat | 19.27 ± 7.52 | 18.55 ± 8.07 | 17.56 ± 6.42 | 18.08 ± 6.62 | 0.75 |
| weight (kg) | | | | | |
| 6-min walking | 312 ± 101 | 347 ± 99 | 319 ± 90 | 338 ± 92 | 0.017 |
| distance (m) | | | | | |
| body water | 62.5 ± 7.8 | 61.7 ± 7.7 | 60.7 ± 8.4 | 61.3 ± 8.2 | 0.88 |
| weight | | | | | |
| percentage (%) | | | | | |

P value is a statistically significant difference between the two groups.

### 2.10 The Method Applied to Elders

In the study, 31 elders were eligible to participate. They must meet the following criteria: (1) age≥75 years old; (2) creatinine level (kidney function)<1.5 g/dl; (3) having walking ability; (4) walking as a rehabilitation twice a day for 30 mins; (5) in addition to daily basic nutritional needs, daily supplying Histidine (1.0 g), Leucine (5.25 g), Isoleucine (1.2 g), Valine (2.25 g); (6) using body comosition scale (OSERIO, Taiwan) to measure body weight, body fat, muscle and body water weight; (7) dietary advice and intervention by the same dietitian; (8) nutritional status at B-C.

According to the method of digitizing nutritional status, muscle turnover, and risk assessment, the elders were divided into two groups (as shown in Table 5), the risk levels of the elders in Group I were A0, A1, A2, and A-1. The risk levels of the elders in Group II were A-4, A-5, A5, and A6. The clinical presentation in the two groups was the same, such as age, gender, body weight, muscle weight and 6-min walking distance, without statistically significant differences. The differences between the two groups could be only distinguished by the method of digitizing nutritional status, muscle turnover, and risk assessment. The elders in Group I were all at low risk as assessed by the method of digitizing nutritional status, muscle turnover, and risk assessment. The elders in Group II were all at high risk as assessed by the method of digitizing nutritional status, muscle turnover, and risk assessment. The elders in the two groups were managed blindly by the same dietitian in the situation of without knowing the results of the method of digitizing nutritional status, muscle turnover, and risk assessment. They accepted the same amount of rehabilitation exercise, and took the same amount of additional amino acids. As shown in Table 5, only the elders of Group I showed a statistically significant improvement in muscle weight and 6-min walking distance after one month. These results validate that the method of digitizing nutritional status, muscle turnover, and risk assessment can effectively identify the elders who have a potential for muscle growth and better rehabilitation results, and thus improve their quality of life.

TABLE 5
Significant differences in muscle growth, rehabilitation results and bodily function after the same
nutrition and rehabilitation interventions are noted between the low risk (Group I) and the high risk
(Group II) elderly patients defined by the method of digitizing nutritional status, muscle turnover,
and risk assessment is applied to patients suffering from various types of chronic diseases

| | Group I (16 elders) | | Group II (15 elders) | | |
|---|---|---|---|---|---|
| | At beginning | After two weeks | At beginning | After two weeks | P value |
| nutrition status | B-C | | B-C | | |
| at beginning | | | | | |
| risk area | A0, A1, A2, A-1 | | A-4, A-5, A5, A6 | | |
| male (%) | 12 (75) | | 11 (73.3) | | 0.91 |
| age (years old) | 76.9 ± 1.5 | | 76.8 ± 1.4 | | 0.97 |
| body weight (kg) | 52.92 ± 10.3 | 53.41 ± 10.1 | 55.33 ± 9.7 | 55.52 ± 9.9 | 0.82 |
| muscle weight (kg) | 22.19 ± 5.01 | 23.42 ± 4.75 | 24.08 ± 4.22 | 23.56 ± 4.37 | 0.03 |
| body fat | 11.5 ± 6.3 | 10.8 ± 6.7 | 11.5 ± 7.2 | 12.2 ± 7.7 | 0.25 |
| weight (kg) | | | | | |
| 6-min walking | 165 ± 57 | 223 ± 72 | 172 ± 61 | 194 ± 59 | 0.02 |
| distance (m) | | | | | |
| body water | 56.3 ± 6.5 | 55.4 ± 6.7 | 59.1 ± 7.7 | 58.7 ± 7.2 | 0.73 |
| weight | | | | | |
| percentage (%) | | | | | |

P value is a statistically significant difference between the two groups.

In summary, the present invention provides a method of digitizing nutritional status, muscle turnover, and risk assessment using the formulas containing four amino acids, which are histidine, leucine, ornithine and phenylalanine. The method of present invention can provide a score regarding personalized nutritional status, muscle turnover, and risk assessment to understand the effect of nutrition intervention, assist muscle growth and the effect of rehabilitation training, and improve patients' quality of life and bodily function. Therefore, the method of the present invention can be applied to determining the digitized staging of disease in aging, cancer, chronic obstructive pulmonary disease, end stage of renal disease, chronic kidney disease and cardiovascular disease; also applied to metabolism assessment, recovering from a critical illness, recovering after surgery and wound healing, muscle growth, monitoring improvement or worsening of disease, rehabilitation results, and assessing nutritional status for healthy individuals.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A method of digitizing nutritional status, muscle turnover, and risk assessment in a subject, comprising the steps of:
(a) measuring the level of an amino acid selected from the group consisting of Histidine, Leucine, Ornithine and Phenylalanine in a biological sample of the subject using a detecting method;
(b) calculating nutritional status and risk scores of the subject using formulas as follows:
(1) Formula 1: Histidine level/Phenylalanine level;
(2) Formula 2: (−19.265 to −15.763)×(the value of Formula 1)+(0.059 to 073)×Ornithine level+(18.776 to 22.948);
(3) Formula 3: the value of Formula 3 calculated based on gender is corrected Ornithine level (Oc):
Male:
if Leucine level≤[(mean of Leucine level in normal male blood)−(standard deviation of Leucine level in normal male blood)],
Oc=[Ornithine level×[(mean of Leucine level in normal male blood)−(standard deviation of Leucine level in normal male blood)]]/Leucine level;
if Leucine level>[(mean of Leucine level in normal male blood)−(standard deviation of Leucine level in normal male blood)],
Oc=Ornithine level;
Female:
if Leucine≤[(mean of Leucine level in normal female blood)−(standard deviation of Leucine level in normal female blood)],
Oc=[Ornithine level×[(mean of Leucine level in normal female blood)−(standard deviation of Leucine level in normal female blood)]]/Leucine level;
if Leucine level>[(mean of Leucine level in normal female blood)−(standard deviation of Leucine level in normal female blood)],
Oc=Ornithine level;
(4) Formula 4: the value of Formula 4 calculated based on gender is corrected Phenylalanine level (Pc):
Male:
if Leucine level≤[(mean of Leucine level in normal male blood)−-(standard deviation of Leucine level in normal male blood)],
Pc=[Phenylalanine level×[(mean of Leucine level in normal male blood)−(standard deviation of Leucine level in normal male blood)]]/Leucine level;
if Leucine level>[(mean of Leucine level in normal male blood)−(standard deviation of Leucine level in normal male blood)],
Pc=Phenylalanine level;
Female:
if Leucine level≤[(mean of Leucine level in normal female blood)−(standard deviation of Leucine level in normal female blood)],
Pc=[Phenylalanine level×[(mean of Leucine level in normal female blood)−(standard deviation of Leucine level in normal female blood)]]/Leucine level;
if Leucine level>[(mean of Leucine level in normal female blood)−(standard deviation of Leucine level in normal female blood)],
Pc=Phenylalanine level; and
(5) Formula 5=−[(−1.414 to −1.157)×Ornithine level/(Leucine level+Histidine level−Phenylalanine level)+(0.0063 to 0.0077)×(the value of Formula 3)]×(9 to 11),
(c) interpreting the nutritional status of the subject,
wherein the value obtained from Formulas 1 and 2 is the nutritional status score, which is compared with the reference value at x-axis of the map of digitizing nutritional status, muscle turnover, and risk assessment, the scale of the nutritional status score is divided as N, A, B, C, and D, N representing normal, A representing early abnormal, B representing significantly abnormal but no symptom, C representing very abnormal, D representing extremely abnormal; the numerical range of N is calculated by a normal subject, and then the numerical range of A, B, C and D are separately defined based on female and male,
Female:
the lower limit of 95% confidence interval of N≤N≤the upper limit of 95% confidence interval of N;
the lower limit of 95% confidence interval of N+4.42≤A≤the lower limit of 95% confidence interval of N+5.96;
the lower limit of 95% confidence interval of N+7.06≤B≤the lower limit of 95% confidence interval of N+9.01;
the lower limit of 95% confidence interval of N+11.75≤C≤the lower limit of 95% confidence interval of N+13.52;
the lower limit of 95% confidence interval of N+18.07≤D≤the lower limit of 95% confidence interval of N+23.66;
Male:
the lower limit of 95% confidence interval of N≤N≤the upper limit of 95% confidence interval of N;
the lower limit of 95% confidence interval of N+3.3≤A≤the lower limit of 95% confidence interval of N+4.46;
the lower limit of 95% confidence interval of N+6.96≤B≤the lower limit of 95% confidence interval of N+7.64;
the lower limit of 95% confidence interval of N+11.36≤C≤the lower limit of 95% confidence interval of N+12.35;
the lower limit of 95% confidence interval of N+18.04≤D≤the lower limit of 95% confidence interval of N+20.77;
the value obtained from the combination of the concentration of Leucine and Formulas 3 to 5 is the risk score, which is compared with the reference value at y-axis of the map of digitizing nutritional status, muscle turnover, and risk assessment to assess risk level from A0 to A8 or from A0 to A-5, which represents gradually higher risks; A0 represents lowest-risk, A8 and A-5 represents highest-risk.

2. The method of claim 1, wherein the Formula 4 further determines the level of muscle lysis.

3. The method of claim 1, wherein the Formula 3 further determines liver metabolic function.

4. The method of claim 1, wherein the Formula 1 further determines muscle turnover.

5. The method of claim 1, wherein the Formula 5 further determines metabolite loading to liver.

6. The method of claim 1, wherein the biological sample of the step (a) is blood, plasma, serum, red blood cells or urine.

7. The method of claim 1, wherein the subject of the step (a) is a patient suffering from a disease or a healthy normal person.

8. The method of claim 7, wherein the disease is aging, cancer, chronic diseases, severe diseases or cardiovascular diseases.

9. The method of claim 8, wherein the chronic disease is chronic obstructive pulmonary disease (COPD), end stage of renal disease (ESRD) or chronic kidney disease (CKD).

10. The method of claim 1, wherein the detecting method of the step (a) is a mass spectrometry (MS), liquid chromatograph (LC), micro-scale capillary electrophoresis or high performance capillary electrophoresis.

11. The method of claim 1, wherein adjusting the subject's nutrition intervention and lifestyle according to result of the step (c).

12. The method of claim 11, wherein the step (a) to the step (c) are done again after adjusting the nutrition intervention and lifestyle.

13. The method of claim 1, wherein the step (a) further comprises to measure body fat, muscle weight, body water weight, body weight, daily dietary water intake of the subject.

14. The method of claim 1, wherein the risk assessment comprises to assess the risk of death or hospitalization due to condition worsening.

15. The method of claim 1, wherein the values obtained from Formulas 1 to 5 calculated based on gender are compared with the reference value at x-axis and y-axis of the map of digitizing nutritional status, muscle turnover, and risk assessment.

16. A kit for digitizing nutritional status, muscle turnover, and risk assessment, which comprises: Histidine, Leucine, Ornithine and Phenylalanine.
